(19) United States
(12) Patent Application Publication
(10) Pub. No.: US 2024/0149036 A1
(43) Pub. Date: May 9, 2024

(54) Conformable Ultrasound Patch For Cavitation Enchanced Transdermal Drug Delivery

(71) Applicant: Massachusetts Institute of Technology, Cambridge, MA (US)

(72) Inventors: Canan DAGDEVIREN, Cambridge, MA (US); Aastha SHAH, Cambridge, MA (US)

(73) Assignee: Massachusetts Institute of Technology, Cambridge, MA (US)

(21) Appl. No.: 18/500,479

(22) Filed: Nov. 2, 2023

Related U.S. Application Data
(60) Provisional application No. 63/422,466, filed on Nov. 4, 2022.
(60) Provisional application No. 63/496,138, filed on Apr. 14, 2023.

(51) Int. Cl. A61M 37/00; A61N 7/00; B06B 1/06

(52) U.S. Cl. CPC A61M 37/0092; A61N 7/00; B06B 1/0629; B06B 1/0651; B06B 2201/76

(57) ABSTRACT

Described herein is an apparatus, comprising a substrate having a top surface and a bottom surface. In an embodiment, the apparatus comprises one or more piezoelectric transducers having a diameter of about 10 mm and a thickness of about 2 mm, embedded in the substrate. In an embodiment, the apparatus comprises one or more cavitation chambers having a depth of about 1 mm, each of the one or more cavitation chambers disposed between respective ones of the one or more piezoelectric transducers and the top surface of the substrate, wherein the one or more piezoelectric transducers generate vibrations within a frequency range about 20 kHz to about 1 MHz in the cavitation chambers that cause a substance stored in the cavitation chambers to be forcibly moved through the top surface of the substrate.

### CROSS REFERENCE SECTION

This application claims the benefit of U.S. Provisional Application No. 63/496,138 filed on Apr. 14, 2023 and U.S. Provisional Application No. 63/422,466 filed on Nov. 4, 2022 which applications are hereby incorporated by reference in their entireties.

### BACKGROUND

There is a desire for safe and effective devices and methods to increase transdermal absorption of therapeutic cosmeceuticals. Transdermal drug delivery offers an attractive alternative to conventional drug delivery methods of intravenous and oral administration due to the non-invasiveness, topical administration method, steady dose rate, and convenience. A number of methods of transdermal drug delivery have been developed, including sonophoresis, which is the use of ultrasound for the delivery of drugs through the skin. However, permeation of small-molecule drugs is limited by the innate barrier function of the different layers of the skin, such as the tightly packed lipid bilayers of the stratum corneum.

### SUMMARY

Disclosed herein is an apparatus, comprising a substrate having a top surface and a bottom surface. In some embodiments, the apparatus comprises one or more piezoelectric transducers having a diameter of about 10 mm and a thickness of about 2 mm, embedded in the substrate. In some embodiments, the apparatus comprises one or more cavitation chambers having a depth of about 1 mm, each of the one or more cavitation chambers disposed between respective ones of the one or more piezoelectric transducers and the top surface of the substrate, wherein the one or more piezoelectric transducers generate vibrations within a frequency range about 20 kHz to about 1 MHz in the cavitation chambers that cause a substance stored in the cavitation chambers to be forcibly moved through the top surface of the substrate.

Disclosed herein is an apparatus, comprising a substrate having a top surface and a bottom surface. In some embodiments, the apparatus comprises two or more piezoelectric transducers having a diameter of about 10 mm and a thickness of about 2 mm, embedded in the substrate and positioned 10 mm or less away from each other. In some embodiments, the apparatus comprises two or more cavitation chambers having a depth of about 1 mm, each of the two or more cavitation chambers disposed between a respective ones of the two or more piezoelectric transducers and the top surface of the substrate, wherein the two or more piezoelectric transducers generate vibrations within a frequency range about 20 kHz to about 1 MHz in the cavitation chambers that cause a substance stored in the cavitation chambers to be forcibly moved through the top surface of the substrate.

According to one aspect of the disclosure, the substrate comprises a bottom substrate having electrical signal paths provided therein and a top substrate having the cavitation chambers provided therein with each of the cavitation chambers arranged to accept a respective one of the transducers. In some embodiments, the substrate is configured to affix to a skin of a user without requiring operator or mechanical fixation. In some embodiments, the frequency range of a first transducer is different than the frequency range of a second transducer.

### DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The manner and process of making and using the disclosed embodiments may be appreciated by reference to the figures of the accompanying drawings. It should be appreciated that the components and structures illustrated in the figures are not necessarily to scale, emphasis instead being placed upon illustrating the principals of the concepts described herein. Like reference numerals designate corresponding parts throughout the different views. Furthermore, embodiments are illustrated by way of example and not limitation in the figures, in which:

FIG. 1A is an exploded perspective view of a conformable ultrasound patch for cavitation enhanced transdermal drug delivery;

FIG. 1B is an enlarged of a portion of the assembled conformable ultrasound patch of FIG. 1A;

FIG. 1C is a top view of a conformable ultrasound patch for cavitation enhanced transdermal drug delivery disposed on a user;

FIG. 2A is an exploded perspective view of a conformable ultrasound patch with an alternative piezoelectric transducer;

FIG. 2B is an enlarged view of the piezoelectric transducer of FIG. 2A;

FIG. 3 is a side cross sectional view of a conformable ultrasound patch for cavitation enhanced transdermal drug delivery disposed on a user's skin;

FIG. 4 is a side cross sectional view of a conformable ultrasound patch with one piezoelectric transducer, including an illustration of the acoustic pressure distribution;

FIG. 5 is a perspective view of a conformable ultrasound patch for cavitation enhanced transdermal drug delivery including four piezoelectric transducers;

FIG. 6 is a side cross sectional view of a conformable ultrasound patch with two piezoelectric transducers, including an illustration of the acoustic pressure distribution;

FIG. 7A is a graph of a gap width between two piezoelectric transducers vs. pressure;

FIG. 7B is a graph of distance between a piezoelectric transducer and a surface of an ultrasound patch vs. pressure;

FIG. 8A is a side cross sectional view of a conformable ultrasound patch with two piezoelectric transducers positioned at a distance of 10 mm apart from one another, including an illustration of the acoustic pressure distribution;

FIG. 8B is a side cross sectional view of a conformable ultrasound patch with two piezoelectric transducers positioned at a distance of 4 mm apart from one another, including an illustration of the acoustic pressure distribution; and

FIG. 8C is a side cross sectional view of a conformable ultrasound patch with two piezoelectric transducers positioned at a distance of 2 mm apart from one another, including an illustration of the acoustic pressure distribution.

### DETAILED DESCRIPTION

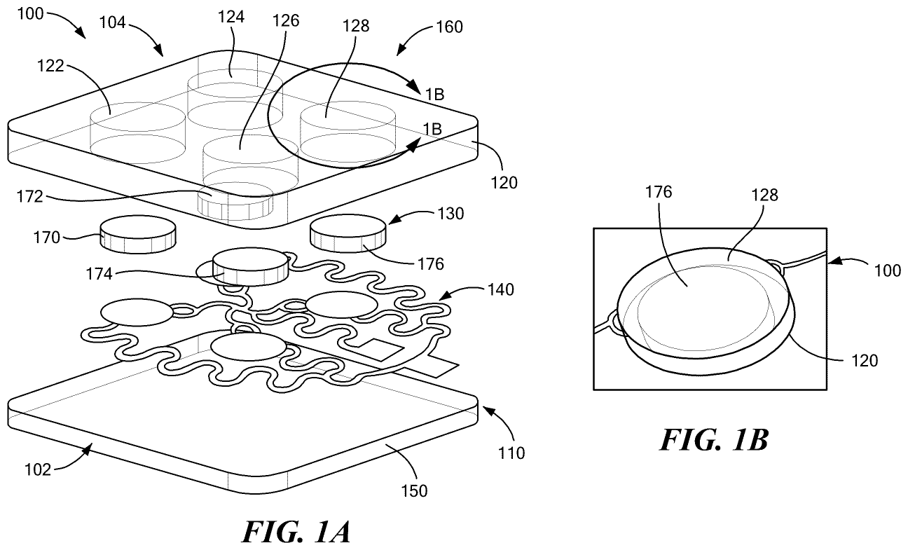

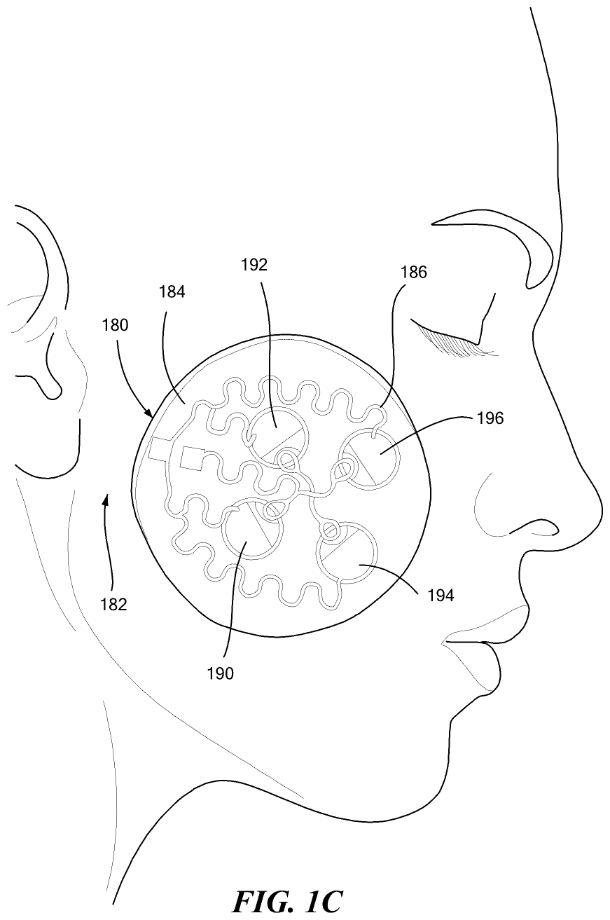

FIG. 1A is an exploded perspective view of a conformable ultrasound patch 100, including a substrate 110, one or more piezoelectric transducers 130, and one or more electrical signal paths 140. The piezoelectric transducers 130 are disposed in one or more cavitation chambers 122, 124, 126, 128 in the substrate 110 and connected to the electrical signal paths 140. The piezoelectric transducers 130 are configured to generate vibrations in the cavitation chambers 122, 124, 126, 128 that cause a substance stored in the cavitation chambers 122, 124, 126, 128 to be forcibly moved through a top surface 104 of the substrate 110. Although this example embodiment includes four (4) piezoelectric transducers, it should be appreciated that any number of piezoelectric transducers may be used. After reading the disclosure provided herein, those of ordinary skill in the art will appreciate how to select the number of piezoelectric transducers to include in a patch intended for use in a particular application.

The substrate 110 is configured to affix to the skin of the user without requiring user or mechanical fixation. Mechanical fixation refers to mechanical structures external to the patch used to hold the patch to the user's skin (e.g. tape or adhesives). The user places the patch onto their skin. The substrate 110 achieves variable conformability and adhesion on curvilinear skin surfaces by varying the thickness and curing time of the polymer.

The piezoelectric transducers 130 include a first piezoelectric transducer 170, a second piezoelectric transducer 172, a third piezoelectric transducer 174, and a fourth piezoelectric transducer 176. The piezoelectric transducers 130 may comprise a crystal, a polymer, or a composite. Examples of suitable piezoelectric materials for the piezoelectric transducers 130 include berlinite (AlPO4), quartz, rochelle salt, topaz, tourmaline-group minerals, gallium orthophosphate (GaP04), langasite (La3Ga5SiOi4), barium titanate (BaTi03), lead titanate (PbTi03), lead zirconate titanate (Pb[ZrxTi1-x]03, 0<x<l) (PZT), lead magnesium niobate (PMN), lead magnesium niobate-lead titanate (PMN-PT), potassium niobate (KNb03), lithium niobate (LiNb03), lithium tantalate (LiTa03), sodium tungstate (Na2W03), zinc oxide (ZnO), sodium potassium niobate ((K,Na)Nb03) (also known as NKN), bismuth ferrite (BiFe03), Sodium niobate (NaNb03), Bismuth titanate (Bi4Ti3O12), sodium bismuth titanate (NBT), polyvinylidene fluoride (PVDF), poly[(vinylidenefluoride-co-trifluoroethylene] [P(VDF-TrFE)3], or combinations thereof. In an embodiment, the piezoelectric transducers may comprise a piezoelectric material comprising a composition of lead indium niobium oxide (PIN), lead magnesium niobium oxide (PMN), and lead titanium oxide (PT), doped with ytterbium and co-doped with bismuth.

Heat generation from the piezoelectric transducers 130 and electrical signal paths 140 within the patch 100 are addressed through various mechanisms. First, by decreasing the duty cycle or total ultrasound application time. A decreased application time enables a decrease in heat generation and a reduction in power consumption. The typical application and operation time for the device is 10 minutes. This time can be decreased based on the desired dosage or penetration depth. Applying a smaller duty cycle may be used to provide a cool-down time for the ultrasound transducer and to control the heat produced within the system. While a decreased duty cycle is desirable from a heating perspective, the application and operation should be sufficiently long to allow the nucleation, growth and collapse of bubbles to take place. The maximum temperature of the patch in use on a user's skin may be less than 47° C. to ensure heat dissipation through the area of the silicone patch. In an embodiment, a 50% duty cycle with 5000 application and operation cycles was used and the maximum temperature at the end of 10 minutes was 47° C.

Second, by adding heat-sinks in the form of a metallic or thermally conductive polymeric mass on the bottom substrate 150 to dissipate the heat to the surrounding air. The heat sinks may be textured metallic blocks with high surface-area-to-volume ratios. The heat sinks are affixed directly to the base of the piezoelectric transducers and facing away from the user's skin towards the bottom surface of the patch.

The piezoelectric transducers 130 are embedded in the substrate 110. The substrate 110 comprises a top substrate 120 and a bottom substrate 150 that once assembled form the substrate 110. The substrate 110 may comprise a polymer or an elastomer. In an embodiment, the piezoelectric transducers 130 are embedded in a polydimethylsiloxane (PDMS) substrate. The PDMS substrate creates a thin-film, waterproof encapsulation for the piezoelectric transducer without dampening the transducer motion or significantly influencing its resonance frequency changes (1% or less change). The PDMS substrate may be formed using a controlled dip-coating method.

Electrical signal paths 140 are disposed in the bottom substrate 150. The electrical signal paths 140 are connected to each of the piezoelectric transducers 130, establishing their electrical connection. In an embodiment, the electrical signal paths 140 may be serpentine metal (Cu) electrodes.

The top substrate 120 comprises one or more cavitation chambers 122, 124, 126, 128 provided in or otherwise disposed along the top surface 104 of the top substrate 120. The piezoelectric transducers 130 are embedded in the top substrate 120 away from the top surface 104 towards a bottom surface 102. The cavitation chambers 122, 124, 126, 128 are formed between the top substrate 120 and the piezoelectric transducers 130. The first piezoelectric transducer 170 is disposed atop a first cavitation chamber 122. The second piezoelectric transducer 172 is disposed atop a second cavitation chamber 124. The third piezoelectric transducer 174 is disposed atop a third cavitation chamber 126. The fourth piezoelectric transducer 176 is disposed atop a fourth cavitation chamber 128. The cavitation chambers 122, 124, 126, 128 have a depth of about 1 mm (+/−0.1 mm).

FIG. 1B is an enlarged view of a portion of the assembled patch 100, including the fourth piezoelectric transducer 176 disposed atop the fourth cavitation chamber 128.

A substance is disposed in the cavitation chambers 122, 124, 126, 128. The substance may be or comprise a liquid. The substance may include small molecule drugs, such as niacinamide (NIA). The radii of the cavitation bubbles formed in the substance in the cavitation chambers 122, 124, 126, 128 is inversely proportional to ultrasound frequency produced by the piezoelectric transducers 130. Acoustic cavitation is defined as the nucleation, growth, oscillation, movement, and collapse of tiny air or vapor bubbles when ultrasound is irradiated to a liquid. Acoustic cavitation can be induced using low frequency sonophoresis (LFS), intermediate frequency sonophoresis (IFS), or high frequency sonophoresis (HFS).

LFS involves frequency of about 20 kHz (+/−5 kHz) to about 100 kHz (+/−10 kHz). IFS involves frequencies of about 100 kHz (+/−10 kHz) to about 1 MHz (+/−10 kHz). HFS involves frequencies over 1 MHz. The frequency regimes enhances skin permeability through different mechanisms, specifically IFS offers an interesting compromise between convective effects and intensity of cavitation. The ranges provided may be used to qualitatively distinguish the types of cavitation action (stable versus inertial) experienced at different frequencies. One or more types of cavitation may simultaneously exist for a given frequency. Stable cavitation is more dominant at HFS, whereas inertial cavitation is more dominant at LFS and IFS.

HFS is used to nucleate cavitation within the stratum corneum, where the bubble size (bubbles with a radius of 20 μm or less) is comparable to the intercellular distance of the lipid bilayers. LFS, which generates larger bubbles (larger bubbles meaning bubbles with a radius of 20 to 200 μm), could be more effective than HFS in drug delivery due to its stronger transient phenomena such as shockwave generation, bubble collapse, and micro-jetting. The cavitation threshold is smaller with LFS, smaller meaning the cavitation threshold is about 60 kPa (+/−10 kPa) at 100 kHz, as compared to about 200 kPa (+/−10 kPa) at 1 MHz. HFS generates higher bubble density and velocities, higher meaning about 10¹⁰ dm⁻³ (+/−1¹⁰ dm⁻³ bubbles) bubbles at 1 MHz, as compared to about 105 dm⁻³ (+/−15 dm⁻³ bubbles) bubbles at 20 kHz, at the expense of electrical power.

HFS treatments can be applied with the transducer in direct contact with the skin, but the enhancement effects implemented by LFS call for a fluid coupling medium, such as a cavitation chamber, in between the transducer and the skin. The cavitation chamber can be challenging to implement, given the multiple systems involved as well as environmental parameters such as: transducer properties; spatial geometry and choice of coupling medium; dissolved gas content; heterogeneity; and separation of target membrane. The 1 mm cavitation chamber disclosed herein provides a large (about 400 μl (+/−100 μl)) field for low or intermediate frequency sonophoresis.

The piezoelectric transducers 130 disposed in the cavitation chambers 122, 124, 126, 128 generate vibrations within a frequency range of about 20 kHz (+/−2 kHz) to about 1 MHz (+/−10 kHz). In some embodiments, the frequency range of the piezoelectric transducers 130 is about 20 kHz (+/−2 kHz) to about 100 kHz (+/−10 kHz). In some embodiments, the frequency range of the piezoelectric transducers 130 is about 100 kHz (+/−10 kHz) to about 1 MHz (+/−10 kHz).

The geometric dimensions of the piezoelectric transducers 130 are selected to achieve a resonance of the crystal in the range of an intermediate ultrasound frequencies (100 kHz-1 MHz). Said range creates sufficient acoustic pressure to drive inertial cavitation. The piezoelectric transducers 130 are driven in the radial mode of operation at the first fundamental mode of vibration. The resonance frequency is a function of the device radius.

In an embodiment, for a circular disc of bulk piezoelectric substrate, the diameter of the disc must be in the range of about 2.20 cm (+/−0.1 cm) to about 0.31 cm (+/−0.1 cm) to ensure a resonance in the range of 100 kHz to 1 MHz. The thickness of the device may be varied between about 100 μm (+/−10 μm) to about 2000 μm (+/−10 μm). In an embodiment, the piezoelectric transducers 130 have a diameter of 10 mm (+/−2 mm) and a thickness of 2 mm (+/−0.5 mm). The piezoelectric transducers may be a disk and may be used with a radial resonance frequency of 220 kHz.

FIG. 1C is top view of a patch 180 affixed to a user 182. The patch 180 comprises a substrate 184, one or more piezoelectric transducers 190, 192, 194, 196, and one or more electrical signal paths 186. The patch 180 comprises a first piezoelectric transducer 190, a second piezoelectric transducer 192, a third piezoelectric transducer 194, and a fourth piezoelectric transducer 196. It will be appreciated by those of ordinary skill in the art, that although four piezoelectric transducers 190, 192, 194, 196 are shown other numbers of piezoelectric transducers are possible including more or fewer piezoelectric transducers.

The substrate 184 may be formed using a three-dimensional (3D) printed polylactic acid mold. The substrate 184 may be disk shaped. Initially, the substrate 184 may be a square with dimensions of about 5 cm (+/−1 cm) width by about 5 cm (+/−1 cm) length. Once formed, the patch 180 may be trimmed to a circle with a diameter of about 5 cm (+/−1 cm). The size of the patch 180 may be adjusted based on the area of the skin targeted. In an embodiment, the patch is demonstrated in an application of niacinamide delivery to the facial cheek, and thus demonstrates delivery over a circular area with a diameter of 5 cm. The patch 180 may be disk shaped.

This design provides sufficient separation between the piezoelectric transducers 190, 192, 194, 196, mitigating any destructive interference of the generated acoustic pressure fields for each and achieving an overall large-area conformal coverage of the skin for drug delivery. The systemic integration of the piezoelectric transducers 190, 192, 194, 196, cavitation chamber, and substance held in the cavitation chamber within a flexible, wearable interface enables a highly effective, localized, and repeatable sonophoresis.

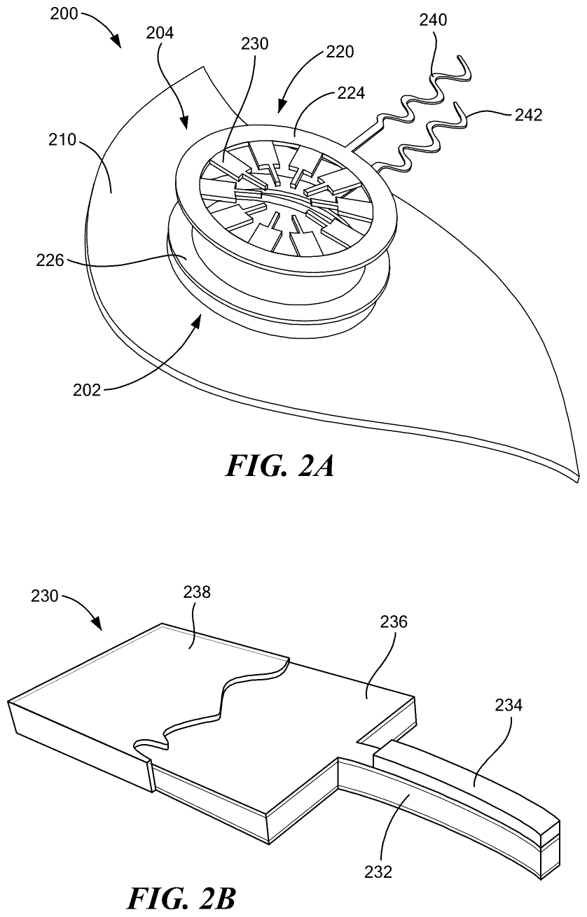

FIG. 2A is an exploded perspective view of a patch 200, which may be similar to patch 100, with an alternative piezoelectric transducer design. The patch 200 comprises a piezoelectric transducer 220 embedded in a substrate 210 with a cavitation chamber 202. The cavitation chamber 202 is disposed below the piezoelectric transducer 220. A first electrode 224 is connected to a first electric signal path 240 and disposed opposite a second electrode 226 that is connected to a second electric signal path 242. One or more piezoelectric cantilevers 230 are shown positioned in the first electrode 224.

FIG. 2B is an enlarged view of the piezoelectric cantilevers 230 of FIG. 2A. Piezoelectric cantilevers 230 includes a piezoelectric unimorph patterned with DRIE 232. Positioned atop the piezoelectric unimorph patterned with DRIE 232 is a gold electrode 236. The gold electrode 236 may be disposed through a sputtering technique, though other techniques may be used. Positioned atop the gold electrode 236 is a thin film PDMS insulation 238. The thin film PDMS insulation 238 may be disposed through a dop-coating technique, though other techniques may be used. Positioned atop the gold electrode 236 is an electroplated passive copper layer 234.

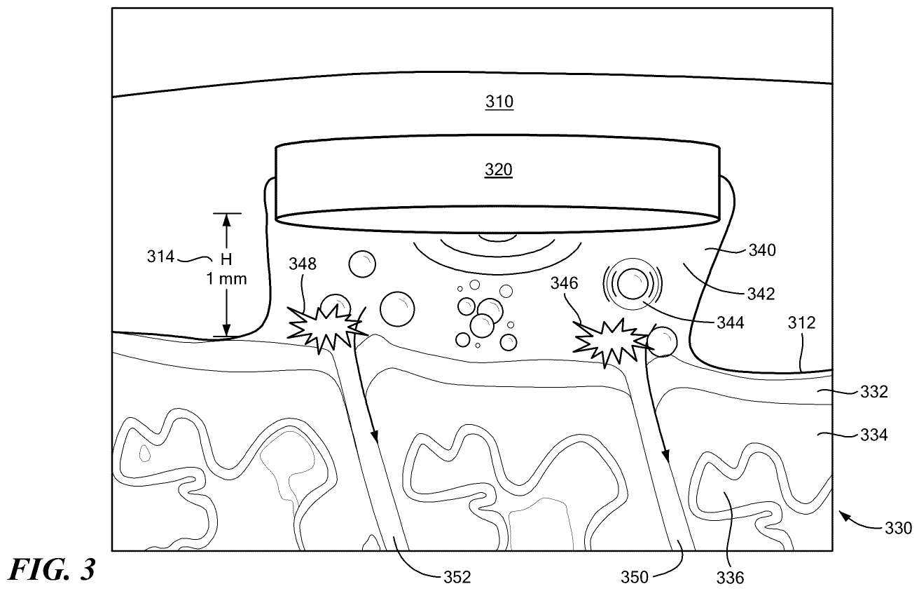

FIG. 3 is a side cross section of a patch 300, including a piezoelectric transducer 320 (like piezoelectric transducers 130) embedded in a substrate 310 (like substrate 110). The substrate 310 is disposed atop a user's skin 330, with a top surface of the substrate 310 in contact with the skin 330. The penetration of drugs and cosmeceuticals through the skin 330 is limited by the innate barrier function of the skin layers to protect the underlying tissue against external substances.

The skin 330 is composed of several layers, including a stratum corneum (SC) 332, an epidermis 334, and a dermis 336. The SC 332 is the outermost layer of the epidermis and is about 5 μm (+/−1 μm) to about 15 μm (+/−1 μm) thick with a structure containing dead keratinocyte cells tightly packed in a continuous structure of lipid bilayers. The thickness of the SC varies at different locations of the user's body. For facial skin, the SC thickness is between about 10 μm (+/−2 μm) to about 30 μm (+/−2 μm). Given this structure, the SC 332 has a low permeability.

A cavitation chamber 340 (like cavitation chambers 122, 124, 126, 128) is disposed between the piezoelectric transducer 320 and the top surface 312, which is positioned on the skin 330. The cavitation chamber 340 has a height 314 of 1 mm, as determined by the space between the piezoelectric transducer 320 and the top surface 312 of the substrate 310. The cavitation chamber 340 provides a 1 mm space for a substance 342, wherein inertial cavitation, convective mixing, and microjet formation can be induced on the surface of the skin 330.

The piezoelectric transducer 320 generates vibrations within the given frequency range in the cavitation chamber 340. These frequencies cause tiny air or vapor bubbles 344 in the substance 342 to collapse 346, 348 when the vibrations are irradiated into the substance 342. Bubbles are nucleated when the peak negative pressure in the ultrasound cycle dips below the cavitation threshold. For liquids at atmospheric pressure, a peak of −101.3 kPa is required to counteract the atmospheric pressure, degassing the dissolved gasses, if present, in the liquid. A lower peak pressure may be sufficient to nucleate cavitation if there are pre-existing bubbles or inclusions in the liquid. Additional substances (e.g. drugs or cosmeceuticals) are dissolved in the fluid medium within which the cavitation action takes place. The substances diffuse through the mechanical pathways created by the bubble collapse through the SC along a standard diffusion gradient.

Said bubble collapse 346, 348 results in microjets 350, 352. A first bubble collapse 346 creates a first microjet 350. A second bubble collapse 348 creates a second microjet 352. The microjets 350, 352 penetrate the surface of the skin 330. This results in the structural disordering of the SC lipids and an increase in the transdermal pathways for the transport of the substance 342 into the skin 330.

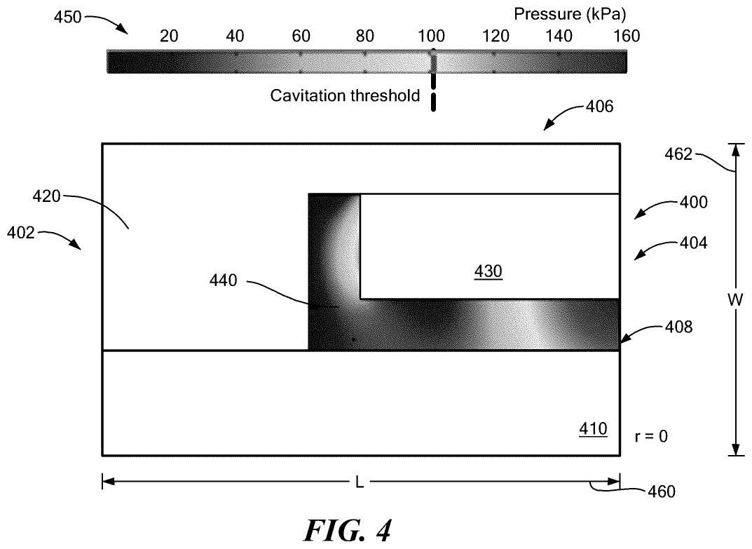

FIG. 4 is a side cross sectional view of a patch 400, including an illustration of the acoustic pressure distribution 450. Disposed atop a user's skin 410, the patch 400 includes a piezoelectric transducer 430 embedded in a substrate 420. The substrate 420 includes a top surface 408 and a bottom surface 406, with a cavitation chamber 440 formed between the top surface 408 and the piezoelectric transducer 430. A substance is contained in the cavitation chamber 440. The patch 400 extends from a first side surface 402 to a second side surface 404 across a length 460, which runs parallel to the top surface 408 and bottom surface 406. Additionally, the patch 400 extends from the top surface 408 and bottom surface 406 across a width 462, which runs parallel to the first side surface 402 and the second side surface 404.

The illustration of the acoustic pressure distribution 450 demonstrates the range of pressure in kilopascals (kPa) from 20 kPa to 160 kPa that may result from the frequency generated by the piezoelectric transducer 430. An acoustic pressure is depicted in the cavitation chamber 440 in the patch 400 by the gradation of color. The strongest acoustic pressure being located closest to the piezoelectric transducer 430 and the weakest being located closest to the top surface 408.

The pressure range generated by the piezoelectric transducer 430 in the substance contained in the cavitation chamber 440 may be different along the length 460 and/or the width 462 of the piezoelectric transducer 430. The pressure range generated by the piezoelectric transducer 430 in the substance contained in the cavitation chamber 440 may increase or decrease along the length 460 and/or the width 462 of the piezoelectric transducer 430. Further, the pressure range generated by the piezoelectric transducer 430 in the substance contained in the cavitation chamber 440 may be the same along the length 460 and/or the width 462 of the piezoelectric transducer 430.

The pressure range generated by the piezoelectric transducer 430 in the substance contained in the cavitation chamber 440 may be defined by the cavitation threshold. In an embodiment, such as that shown in FIG. 4, the cavitation threshold may be 100 kPa. In an embodiment, such as that shown in FIG. 4, the acoustic pressure distribution in the device operating in radial mode at frequency of 212 kHz with an applied voltage of 50 V illustrates an undamped pressure zone (of about 100 kPa (+/−10 kPa)) within the substance in the cavitation chamber, which is enough to nucleate cavitation.

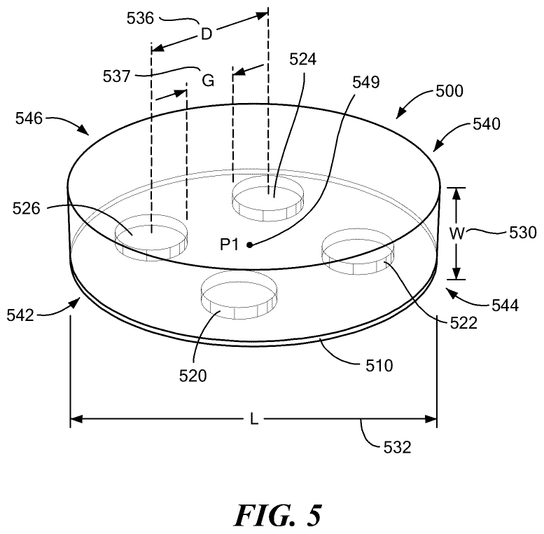

FIG. 5 is a perspective view of a patch 500 including four piezoelectric transducers 520, 522, 524, 526 embedded in a substrate 510. The patch 500 extends across a width 530 and a length 532. The width 530 may be about 4 mm (+/−1 mm). The patch 500 includes a first side 540 opposed to a second side 542 and a third side 544 opposed to a fourth side 546. The first side 540 and the third side 544 run parallel to one another while the second side 542 and the fourth side 546 run parallel to one another. A first pressure point P1 549 is depicted at a center point on the patch 500. The point P1 549 may be 1 mm from the transducers.

A first piezoelectric transducer 520 is positioned towards the second side 542 and the third side 544. A second piezoelectric transducer 522 is positioned towards the third side 544 and the first side 540. A third piezoelectric transducer 524 is positioned towards the first side 540 and the fourth side 546. A fourth piezoelectric transducer 526 is positioned towards fourth side 546 and the second side 542.

The piezoelectric transducers 520, 522, 524, 526 are positioned in rows. In an embodiment, such as that depicted in FIG. 5, the rows are symmetric in a 2×2 array. In an embodiment, the first piezoelectric transducer 520 and the second piezoelectric transducer 522 forming a first row along the third side 544. The third piezoelectric transducer 524 and fourth piezoelectric transducer 526 form a second row along the fourth side 546. In an alternative embodiment, the first piezoelectric transducer 520 and the third piezoelectric transducer 524 forming a first row along the first side 540. The second first piezoelectric transducer 522 and the fourth piezoelectric transducer 526 forming a second row along the second side 542.

The piezoelectric transducers 520, 522, 524, 526 are positioned at a distance 536 away from one another in their respective rows. The distance 536 is measured from the center point of the piezoelectric transducer to the center point of a consecutive piezoelectric transducer in the same row. Following, the fourth piezoelectric transducer 526 is positioned a distance 536 away from the third piezoelectric transducer 524 on the second row along the fourth side 546. The piezoelectric transducers may have a distance of 10 mm or less, 4 mm or less, or 2 mm away from one another. The piezoelectric transducers 520, 522, 524, 526 have a gap 537 between one another in their respective rows. The gap 537 is measured from an edge of the piezoelectric transducer to an edge of a consecutive piezoelectric transducer in the same row. Following, there is a gap 537 between the fourth piezoelectric transducer 526 and the third piezoelectric transducer 524 on the second row along the fourth side 546. The piezoelectric transducers may have a gap of 10 mm or less between one another.

The frequency range of the piezoelectric transducer may be different than the other piezoelectric transducers in the patch 500. The frequency range of the first piezoelectric transducer 520 may be different or the same as the second piezoelectric transducer 522. Further, the frequency range of the first piezoelectric transducer 520 and the second piezoelectric transducer 522 may be different or the same as the frequency range of the fourth piezoelectric transducer 526 and the third piezoelectric transducer 524.

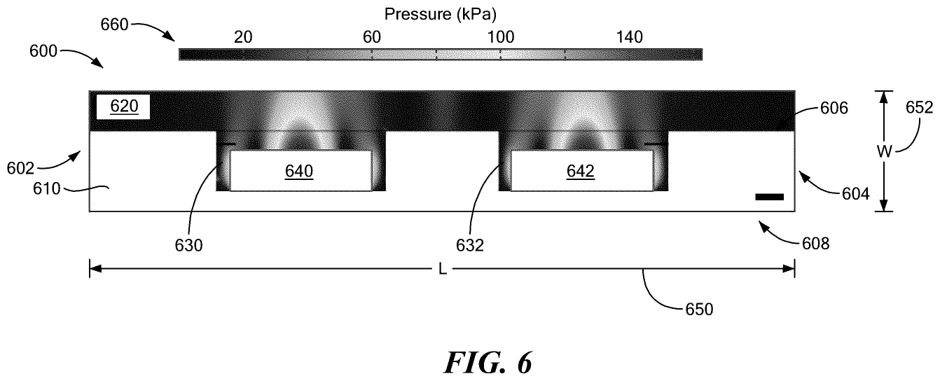

FIG. 6 is a side cross-sectional view of a patch 600, including an illustration of the acoustic pressure distribution 660. Disposed atop a user's skin 620, the patch 600 includes two piezoelectric transducers 640, 642 embedded in a substrate 610. The substrate 610 includes a top surface 606 and a bottom surface 608, with two cavitation chambers 630, 632 formed between the top surface 606 and the piezoelectric transducers 640, 642. A substance is contained in the cavitation chamber 630, 632. The patch 600 extends from a first side surface 602 to a second side surface 604 across a length 650, which runs parallel to the top surface 606 and bottom surface 608. Additionally, the patch 600 extends from the top surface 608 and bottom surface 608 across the width 652, which runs parallel to the first side surface 602 and the second side surface 604. A first piezoelectric transducer 640 is disposed in a first cavitation chamber 630. A second piezoelectric transducer 642 is disposed in a second cavitation chamber 632.

The illustration of the acoustic pressure distribution 660 demonstrates the range of pressure in kilopascals (kPa) from 0 kPa to 150 kPa that may result from the frequency generated by the piezoelectric transducers 640, 642. The distance between the piezoelectric transducers 640, 642 may be established to ensure the pressure given off by each piezoelectric transducers 640, 642 does not affect the other. An acoustic pressure is depicted in the cavitation chambers 630, 632 in the patch 600 by the gradation of color. The strongest acoustic pressure being located closest to the piezoelectric transducers 640, 642 and the weakest being located closest to the top surface 606.

The pressure range generated by the piezoelectric transducers 640, 642 in the substance contained in the cavitation chambers 630, 632 may be different along the length 650 and/or the width 652 of each of the transducers 640, 642. The pressure range generated by each of the piezoelectric transducers 640, 642 in the substance contained in the cavitation chambers 630, 632 may increase or decrease along the length 650 and/or the width 652 of each of the piezoelectric transducers 640, 642. Further, the pressure range generated by each of the piezoelectric transducers 640, 642 in the substance contained in the cavitation chambers 630, 632 may be the same along the length 650 and/or the width 652 of each of the piezoelectric transducers 640, 642. The pressure range generated by each of the piezoelectric transducers 640, 642 may be the same or different. Interactions of the pressure given off by each piezoelectric transducers 640, 642 may arise with smaller transducer spacings.

The piezoelectric transducers 640, 642 shown in FIG. 6 are placed at a distance of 20 mm away from each other (as measured by the center point of the piezoelectric transducers 640, 642, in accordance with the distance shown in FIG. 5) and driven in parallel at 50 V. The pressure shown suggests there is little acoustic interaction between the two.

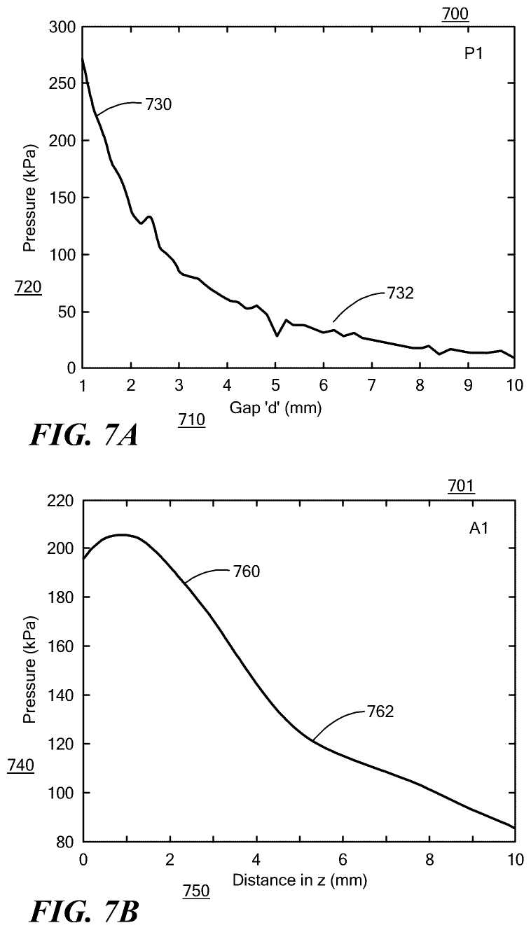

FIG. 7A is graph 700 of the gap 710 between two piezoelectric transducers in mm vs. the pressure 720 in kPa. The gap 710 is measured from an edge of the piezoelectric transducer to an edge of a consecutive piezoelectric transducer in the same row, in accordance with the gap 537 in FIG. 5. The pressure 720 is collected at a pressure point, such as the point P1 549 in FIG. 5. The graph 700 indicates the pressure is higher when the gap is smaller, as indicated by point 730. Further, the pressure is lower when the gap is higher, as indicated by point 732. While varying the gap distance from about 0.5 mm (+/−0.1 mm) to about 5 mm (+/−0.1 mm).

FIG. 7B is a graph 701 of the distance 740 between the piezoelectric transducer and the top surface of the substrate in mm vs. the pressure 740 in kPa. The graph 701 of the distance 750 between the piezoelectric transducer and the top surface in mm vs. the pressure 740 in kPa. The distance 740 is measured by the space between the piezoelectric transducer and the top surface of the substrate, in accordance with the height 314 in FIG. 3. The graph 701 indicates the pressure is higher when the distance is smaller, as indicated by point 760. Further, the pressure is lower when the distance is higher, as indicated by point 762. The acoustic pressure 740 drops off with increasing distance 750 away from the transducer surface. The graph 701 indicates that the pressure 740 peaks close to 1 mm from the surface and then starts to drop off. Accordingly, having a 1 mm cavitation chamber is sufficient to build high acoustic pressures to nucleate cavitation.

The graphs 700, 701 disclose the effect of constructive or destructive influence of the acoustic pressure fields created by each piezoelectric transducer at the center of the patch. Enabling the creation of high acoustic pressure zones in regions other than directly below the piezoelectric transducers, increasing the area of skin treated by the ultrasound energy.

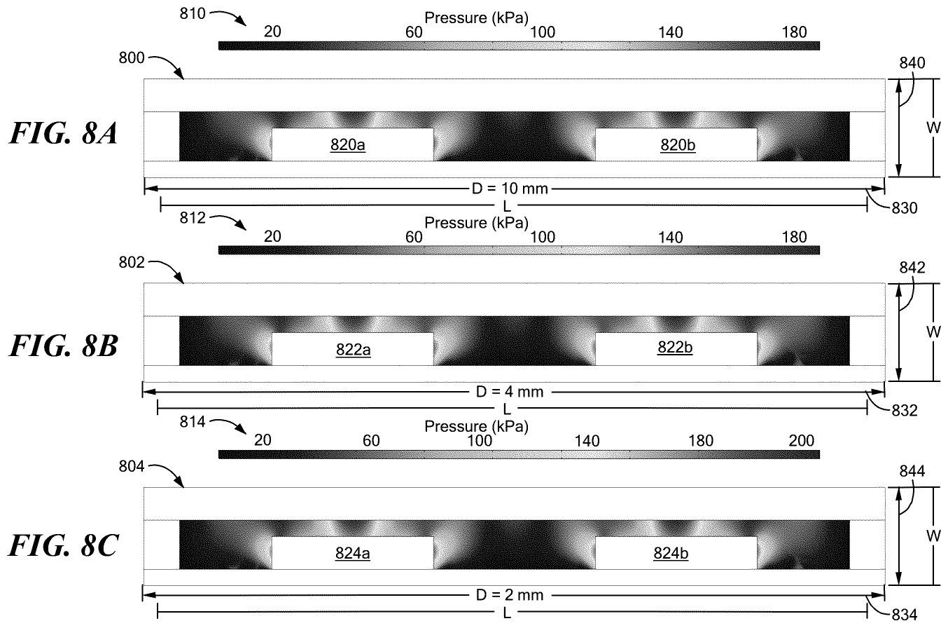

FIGS. 8A, 8B, 8C are a side cross sectional view of conformable ultrasound patches 800, 802, 804, including an illustration of the acoustic pressure distributions 810, 812, 814. The conformable ultrasound patches 800, 802, 804 and acoustic pressure distributions 810, 812, 814 may be similar to the patch 600 and the illustration of the acoustic pressure distribution 660 of FIG. 6.

FIG. 8A is a side cross sectional view of a first patch 800 with two piezoelectric transducers 820a, 820b positioned at a distance of 10 mm apart from one another, including an illustration of the acoustic pressure distribution 810. The first patch 800 comprises a first piezoelectric transducer 820a positioned adjacent to a second piezoelectric transducer 820b embedded in a substrate along a length 830 of the first patch 800. The illustration of the acoustic pressure distribution 810 demonstrates the range of pressure in kilopascals (kPa) from 0 kPa to 180 kPa that may result from the frequency generated by the piezoelectric transducers 820a, 820b. The piezoelectric transducers 820a, 820b are positioned at a distance (as measured by the center point of the piezoelectric transducers 820a, 820b, in accordance with the distance shown in FIG. 5) of 10 mm away from each other. The distance between the piezoelectric transducers 820a, 820b may be established to ensure the pressure given off by each piezoelectric transducers 820a, 820b does not affect the other. An acoustic pressure is depicted along a width 840 and the length 830 of the first patch 800 by the gradation of color. The strongest acoustic pressure being located closest to the piezoelectric transducers 820a, 820b.

FIG. 8B is a side cross sectional view of a second patch 802 with two piezoelectric transducers 822a, 822b positioned at a distance of 4 mm apart from one another, including an illustration of the acoustic pressure distribution 812. The second patch 802 comprises a first piezoelectric transducer 822a positioned adjacent to a second piezoelectric transducer 822b embedded in a substrate along a length 832 of the first patch 802. The illustration of the acoustic pressure distribution 812 demonstrates the range of pressure in kilopascals (kPa) from 0 kPa to 180 kPa that may result from the frequency generated by the piezoelectric transducers 822a, 822b. The piezoelectric transducers 822a, 822b are positioned at a distance (as measured by the center point of the piezoelectric transducers 822a, 822b, in accordance with the distance shown in FIG. 5) of 4 mm away from each other. The distance between the piezoelectric transducers 822a, 822b may be established to ensure the pressure given off by each piezoelectric transducers 822a, 822b does not affect the other. An acoustic pressure is depicted along a width 842 and the length 832 of the second patch 802 by the gradation of color. The strongest acoustic pressure being located closest to the piezoelectric transducers 822a, 822b.

FIG. 8C is a side cross sectional view of a second patch 804 with two piezoelectric transducers 824a, 824b positioned at a distance of 2 mm apart from one another, including an illustration of the acoustic pressure distribution 814. A third patch 804 comprises a first piezoelectric transducer 824a positioned adjacent to a second piezoelectric transducer 824b embedded in a substrate along a length 834 of the first patch 804. The illustration of the acoustic pressure distribution 814 demonstrates the range of pressure in kilopascals (kPa) from 0 kPa to 180 kPa that may result from the frequency generated by the piezoelectric transducers 824a, 824b. The piezoelectric transducers 824a, 824b are positioned at a distance (as measured by the center point of the piezoelectric transducers 824a, 824b, in accordance with the distance shown in FIG. 5) of 2 mm away from each other. The distance between the piezoelectric transducers 824a, 824b may be established to ensure the pressure given off by each piezoelectric transducers 824a, 824b does not affect the other. An acoustic pressure is depicted along a width 844 and the length 834 of the third patch 804 by the gradation of color. The strongest acoustic pressure being located closest to the piezoelectric transducers 824a, 824b.

The effect of the acoustic pressure fields can be visualized clearly in the patches 800, 802, 804. The pressure decreases when the piezoelectric transducers are positioned farther from one another, as seen by the color graduation indicating the pressure given off in patch 800. Further demonstrated by the decrease in pressure 720 when the gap 710 increases, as indicated by point 732 in graph 700. The pressure increases when the piezoelectric transducers are positioned closer to one another, as seen by the color graduation indicating the pressure given off in patch 804. Further demonstrated by the decrease in pressure 720 when the gap 710 decreases, as indicated by point 730 in graph 700.

### Example—Fabrication of the Conformable Ultrasound Patch

The piezoelectric discs were dip-coated in polydimethylsiloxane (PDMS) and cured at 100 C for 1 hour. The electrodes were covered with a piece of tape that was removed after the dip-coating to expose the pads for connection to the circuit. For electrical connections to the device, photolithography was used to fabricate the copper interconnects on a 36 μm thick copper foil. Several drops of photoresist were dispensed onto the copper foil attached on a silicon wafer and the substrate was spun at 1500 rpm for 45 seconds using a spin coater. The substrate was then baked at 95° C. for 2 minutes followed by 110° C. for 2 minutes on a hotplate. To define the serpentine pattern, the substrate was exposed to ultraviolet (UV) at 403 nm under a transparency mask for 15 seconds, developed using the developer, and baked at 120° C. for 5 minutes. Next, the substrate was placed in a bath of copper etchant at 120° C. for 1 hour to remove the exposed copper. Finally, to dissolve the underlying adhesive, the substrate was immersed in a bath of stripper at 120° C. until the serpentine pattern was lifted off. The electrode was then dried off and ready for subsequent assembly.

To assemble the array, the coated piezoelectric transducer elements were positioned in a three dimensional (3D) printed poly-lactic acid filament mold with a 20 mm distance between adjacent elements. The serpentine electrode was then positioned in the mold and attached to the electrodes on the back side of the piezoelectric transducer elements with solder paste. Copper wires were then soldered to the top side of the copper foil for electrical connection. The parts of PDMS were mixed at 10:1 weight ratio thoroughly, degassed in a vacuum chamber, and poured into the mold to a thickness of 4 mm to completely encapsulate the piezoelectric transducer elements and the serpentine interconnects. In an embodiment, the device employs 10:1 PDMS (polydimethylsiloxane) which is cured for 24 hours at 25° C. with a uniform thickness of 4 mm.

Although reference is made herein to particular materials, it is appreciated that other materials having similar functional and/or structural properties may be substituted where appropriate, and that a person having ordinary skill in the art would understand how to select such materials and incorporate them into embodiments of the concepts, techniques, and structures set forth herein without deviating from the scope of those teachings.

Various embodiments of the concepts, systems, devices, structures and techniques sought to be protected are described herein with reference to the related drawings. Alternative embodiments can be devised without departing from the scope of the concepts, systems, devices, structures and techniques described herein. It is noted that various connections and positional relationships (e.g., over, below, adjacent, etc.) are set forth between elements in the following description and in the drawings. These connections and/or positional relationships, unless specified otherwise, can be direct or indirect, and the described concepts, systems, devices, structures and techniques are not intended to be limiting in this respect. Accordingly, a coupling of entities can refer to either a direct or an indirect coupling, and a positional relationship between entities can be a direct or indirect positional relationship.

As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s). The following definitions and abbreviations are to be used for the interpretation of the claims and the specification. As used herein, the terms “comprises,” “comprising, “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance, or illustration. Any embodiment or design described herein as “exemplary” is not necessarily to be construed as preferred or advantageous over other embodiments or designs. The term “one or more” is understood to include any integer number greater than or equal to one, i.e. one, two, three, four, etc. The terms “a plurality” are understood to include any integer number greater than or equal to two, i.e. two, three, four, five, etc. The term “connection” can include an indirect “connection” and a direct “connection.”

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal, “top,” “bottom,” and derivatives thereof shall relate to the described structures and methods, as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element. The term “direct contact” means that a first element, such as a first structure, and a second element, such as a second structure, are connected without any intermediary elements.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. An apparatus, comprising:
a substrate having a top surface and a bottom surface;
one or more piezoelectric transducers having a diameter of about 10 mm and a thickness of about 2 mm, embedded in the substrate; and
one or more cavitation chambers having a depth of about 1 mm, each of the one or more cavitation chambers disposed between respective ones of the one or more piezoelectric transducers and the top surface of the substrate, wherein the one or more piezoelectric transducers generate vibrations within a frequency range about 20 kHz to about 1 MHz in the cavitation chambers that cause a substance stored in the cavitation chambers to be forcibly moved through the top surface of the substrate.

2. The apparatus of claim 1, wherein the substrate comprises a bottom substrate having electrical signal paths provided therein and a top substrate having the cavitation chambers provided therein with each of the cavitation chambers arranged to accept a respective one of the transducers.

3. The apparatus of claim 1, wherein the substrate comprises a polymer or an elastomer.

4. The apparatus of claim 1, wherein substrate is configured to affix to a skin of a user without requiring operator or mechanical fixation.

5. The apparatus of claim 1, wherein the one or more piezoelectric transducers comprise a crystal, a polymer, or a composite.

6. The apparatus of claim 1, wherein the frequency range is about 20 kHz to about 100 kHz.

7. The apparatus of claim 1, wherein the frequency range is about 100 kHz to about 1 MHz.

8. An apparatus, comprising:
a substrate having a top surface and a bottom surface;
two or more piezoelectric transducers having a diameter of about 10 mm and a thickness of about 2 mm, embedded in the substrate and positioned 10 mm or less away from each other; and
two or more cavitation chambers having a depth of about 1 mm, each of the two or more cavitation chambers disposed between a respective ones of the two or more piezoelectric transducers and the top surface of the substrate, wherein the two or more piezoelectric transducers generate vibrations within a frequency range about 20 kHz to about 1 MHz in the cavitation chambers that cause a substance stored in the cavitation chambers to be forcibly moved through the top surface of the substrate.

9. The apparatus of claim 8, wherein the substrate comprises a bottom substrate having electrical signal paths provided therein and a top substrate having the cavitation chambers provided therein with each of the cavitation chambers arranged to accept a respective one of the transducers.

10. The apparatus of claim 8, wherein the substrate comprises a polymer or an elastomer.

11. The apparatus of claim 8, wherein substrate is configured to affix to a skin of a user without requiring operator or mechanical fixation.

12. The apparatus of claim 8, wherein the one or more piezoelectric transducers comprise a crystal, a polymer, or a composite.

13. The apparatus of claim 8, wherein the frequency range is about 20 kHz to about 100 kHz.

14. The apparatus of claim 8, wherein the frequency range is about 100 kHz to about 1 MHz.

15. The apparatus of claim 8, wherein the frequency range of a first transducer is different than the frequency range of a second transducer.

16. The apparatus of claim 8, wherein the frequency range of a first transducer is higher than the frequency range of a second transducer.

17. The apparatus of claim 8, wherein the two or more piezoelectric transducers are positioned 4 mm or less away from each other.

18. The apparatus of claim 8, wherein the two or more piezoelectric transducers are positioned 2 mm away from each other.

19. The apparatus of claim 8, wherein there are two piezoelectric transducers.

20. The apparatus of claim 8, wherein there are four piezoelectric transducers.
